(19) United States
(12) Patent Application Publication
(10) Pub. No.: US 2024/0033268 A1
(43) Pub. Date: Feb. 1, 2024

(54) COMPOSITIONS FOR A DIETARY SUPPLEMENT TO IMPROVE FOCUS

(71) Applicant: Neurohacker Collective, LLC, Carlsbad, CA (US)

(72) Inventor: Gregory KELLY, Carlsbad, CA (US)

(21) Appl. No.: 17/876,424

(22) Filed: Jul. 28, 2022

(51) Int. Cl. A61K 31/52; A61K 31/13; A61K 31/519; A61K 36/258; A61K 36/37; A61K 9/28

(52) U.S. Cl. CPC A61K 31/52; A61K 31/13; A61K 31/519; A61K 36/258; A61K 36/37; A61K 9/28

(57) ABSTRACT

A composition for improving mental energy. In one embodiment, the composition comprises Caffeine, L-Theanine, L-Ornithine, B-complex vitamins—Thiamine (B1), Riboflavin (B2), Niacinamide (B3), Pantothenic Acid (B5), Pyridoxal 5′-phosphate (B6), Folate (B9), and Methylcobalamin (B12). In certain embodiments, the composition further comprises Celastrus paniculatus Seed Extract, American Ginseng (Panax quinquefolius) Root Extract, or a combination thereof. In other embodiments, the composition further includes Ginger Root Extract, Ginkgo biloba Leaf Extract, or a combination thereof. In certain embodiments, the composition is incorporated into pills. The composition boosts the sense of mental energy during the day, manifesting as increased alertness, attention, focus, mood, and motivation, by promoting brain energy production and a wide range of brain and nervous system functions that support mental energy.

### BACKGROUND OF THE INVENTION

### 1. Field of Invention

The present invention relates to dietary supplements to optimize mental energy, including clarity, memory, and drive.

### 2. Description of Related Art

Mental energy is the capacity to carry out daily living tasks that manifest as feeling energetic, being motivated, and having a greater capacity for focused attention. Mental energy is often regarded as a unidimensional measure of central nervous system arousal, and activation demonstrated in the ability for sustained vigilance. But this is a limited view. A more comprehensive conceptualization of mental energy presents it as a three-dimensional construct consisting of cognition, mood, and motivation. The cognitive dimension of mental energy refers to parameters such as sustained attention or vigilance and the mental effort required to maintain performance during a cognitive task. Mood refers to the spirit of energy, i.e., feelings about the presence of energy or fatigue often referred to as alertness. The motivational dimension refers to the activating force, i.e., the drive to act toward a desired goal and the determination and enthusiasm to accomplish mental tasks.

According to this conceptualization, mental energy is more accurately defined as the ability to perform well on cognitive tasks that require sustained attention, intense feelings of energy, and motivation to accomplish mental tasks. Accordingly, low mental energy or mental fatigue is defined as an inability to maintain performance during the completion of a cognitive task, with feelings of low energy and high fatigue and low motivation to carry out mental tasks.

Low mental energy is a common complaint among adults, occurring in about 25% of the population. Its prevalence is exceptionally high in women, younger working adults, and older individuals. An economic burden is associated with low mental energy driven primarily by healthcare costs and lost productivity. Enhancement of mental energy can increase work productivity, as well as the quality of life. There are currently several dietary supplements and food and beverage products marketed for their potential to enhance mental energy. Sales of energy drinks or supplements have been increasing rapidly and steadily, reflecting a necessity or desire among many individuals to improve their mental energy.

Selected herbs, vitamins, amino acids, and other compounds that qualify as dietary supplements can support certain aspects of mental energy. These ingredients may be found in dietary supplement products as single compound only products (i.e., either Caffeine or L-Theanine, as an example) or as combinations of ingredients (i.e., both Caffeine and L-Theanine). Because of the ingredient(s) chosen, dosage, or the ingredient(s) not included, many of these products are unsuitable for day-long, sustained support of mental energy. For example, caffeine is the most widely used supplement for brain performance and the ingredient most commonly found in mental energy products. Caffeine shows a great capacity to promote alertness, sustained attention, concentration, and motivation. However, these effects often come with costs such as jitteriness. They can be brief, lasting several hours at most, followed by an energy crash often accompanied by tension and irritability. Another problem with energy products is that many are highly caffeinated and loaded with sugar with several potential adverse effects, including cardiovascular and metabolic side effects. There is a need for a product that safely supports mental energy without relying so heavily on caffeine or sugar.

Some evidence shows that products combining lower doses of caffeine with other bioactive ingredients have synergistic effects that exceed those of caffeine alone. The reason for the limited impact of caffeine on mental energy is, in part, the fact that it is a unidimensional approach to a multidimensional capacity. Several biological processes and neurological functions have a potential role in maintaining and improving mental energy and are not satisfactorily addressed by a single ingredient. For example, mental energy's cognitive, mood, and motivational dimensions are sustained by different neurotransmitter systems and brain regions that, albeit having independent roles in supporting mental energy, are also interconnected in this process. Ideally, they should be targeted as comprehensively as possible. Notably, a bioenergetic element in mental energy also refers to the capacity to sustain mental activity biochemically. The neuronal activity that underlies mental activity has very high energetic demands. This is made clear by the fact that the brain uses roughly 20% of the daily energy budget for the body, and the more demanding, effortful, or sustained the type of mental activity, the more energy it requires. This must be powered by the oxidative metabolism of dietary carbohydrates, lipids, and proteins, which generates cellular energy as adenosine triphosphate (ATP) through cellular respiration. This is another aspect of mental energy that must be addressed appropriately in a composition that supports mental energy in a sustained and prolonged way.

There are several common problems with current compositions for improving mental energy. A common problem with present compositions is that their effect is too short-lived, often leading to a crash after a few hours. Therefore, what is needed is a composition that, if taken in the morning, can boost mental energy throughout the day. Also, a common problem with current compositions for improving mental energy is that they are unidimensional, lacking the capacity to target different processes that support mental energy. A composition is needed that addresses the multiple dimensions of mental energy, provides comprehensive and sustained support for cognitive performance, and continuously upgrades brain performance with continued use. This need has heretofore remained unsatisfied.

### SUMMARY OF THE INVENTION

The present invention is directed to a dietary supplement designed to enhance mental energy and cognitive performance by boosting the sense of energy during the day, increasing the capacity to exert mental effort, increasing mental alertness, improving attention, enhancing memory, enhancing motivation, and increasing resilience to stress.

In one embodiment of the invention, a dietary supplement composition comprises Caffeine, L-Theanine, L-Ornithine, B-complex vitamins—Thiamine (B1), Riboflavin (B2), Niacinamide (B3), Pantothenic Acid (B5), Pyridoxal 5′-phosphate (B6), Folate (B9), Methylcobalamin (B12). In another embodiment of the present invention, the composition further comprises Celastrus paniculatus Seed Extract, American Ginseng (Panax quinquefolius) Root Extract, or any combination thereof. In another embodiment of the invention, the composition further comprises Ginger Root Extract, Ginkgo biloba Leaf Extract, or any combination thereof.

In an exemplary embodiment of the invention, the composition is integrated into a capsule, a tablet, a chewable tablet, or a pill to be taken orally.

The advantages of the present invention include inducing an overall upregulation of mental energy by supporting alertness, sustained attention, focus, mood, motivation, and brain energy production. Other advantages include neuro-adaptogen support for dealing with stress and increasing mental fatigue resistance, memory support, and long-term brain health support.

The foregoing, and other features and advantages of the invention, will be apparent from the following, more particular description of the preferred embodiments of the invention and the accompanying drawings.

### BRIEF DESCRIPTION OF THE DRAWINGS

For a more complete understanding of the present invention, the objects, and advantages thereof, reference is now made to the ensuing descriptions taken in connection with the accompanying drawings briefly described as follows:

FIG. 1 illustrates a composition according to an embodiment of the invention;

FIG. 2 illustrates a table of exemplary amounts of ingredients, all of the various subsets of which are found in one or more embodiments of the present invention; and

FIG. 3 illustrates a table of exemplary amounts of ingredients, all of the various subsets of which are found in one or more embodiments of the present invention.

### DETAILED DESCRIPTION OF THE EMBODIMENTS

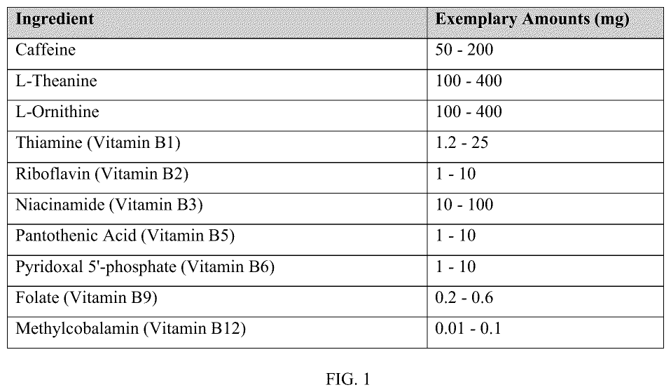

Preferred embodiments of the present invention and their advantages may be understood by referring to FIGS. 1-3. Further features and advantages of the invention, as well as the structure and operation of various embodiments of the invention, are described in detail below. Although the invention is described in the context of a compound, any form, including but not limited to a powder, a pill, a liquid, or another form suitable for various administrations, may be implemented.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention, provided they come within the scope of the appended claims and their equivalents. Reference will now be made in detail to the preferred embodiments of the invention.

In an exemplary embodiment of the present invention and with reference to FIG. 1, a dietary supplement that improves mental energy comprises Caffeine, L-Theanine, L-Ornithine, B-complex vitamins—Thiamine (B1), Riboflavin (B2), Niacinamide (B3), Pantothenic Acid (B5), Pyridoxal 5′-phosphate (B6), Folate (B9), Methylcobalamin (B12). In one embodiment of the invention, the total mg in one dose is 531 mg and is taken in one capsule. Caffeine is the most widely used and possibly studied dietary supplement ingredient for brain performance. It promotes alertness and counters fatigue, particularly for tasks in the complex attention category (e.g., concentration, focus, and reaction speed). Caffeine also produces a sense of motivation and has an uplifting impact on some aspects of mood. L-Theanine is a calming amino acid that naturally occurs in green tea. L-theanine helps adapt to mentally fatiguing or stressful circumstances by promoting alpha brain waves, associated with relaxation, focused attention, mental alertness, and feelings of calm, relaxed energy. L-theanine complements caffeine in supporting a more centered feeling of mental energy, alertness, and focus than caffeine alone. This can manifest as experiencing a sense of flow and enhanced productivity while feeling calmer and less stressed. L-Ornithine is an amino acid that sits at the crossroads of the GABA (Gamma aminobutyric) and glutamate neurotransmitter pathways. L-Ornithine extends the mood and motivation benefits of caffeine. L-ornithine complements caffeine by prolonging the attention-related nootropic responses to caffeine for up to 8 hours while supporting mood, vigor, and willingness to work to a greater degree than caffeine alone. The B-complex family of vitamins is a group of water-soluble vitamins that play important roles in cellular metabolism and energy production. Numerous B vitamins function as precursors of various coenzymes and electron acceptors involved in the pathways of cell energy production. In addition to their role in oxidative metabolism, B vitamins are important for brain function, nerve health, neurotransmitter synthesis and regulation, brain energy metabolism, and antioxidant defenses. B-vitamin deficiencies are associated with mental fatigue and cognitive decline. A sizable proportion of the populations of developed countries have been shown to exhibit B-vitamin deficiencies, and an even larger group may experience inadequacies predisposing them to fatigue. These risks are due, among other factors, to the nutrient profile of modern diets or age-related reductions in vitamin uptake. Thiamine (vitamin B1) concentrates in metabolically active tissues such as the brain. Thiamine is an important nootropic vitamin for producing mental energy from carbohydrates. It supports brain energy production, neuron health, and neurotransmission. Thiamine also supports reaction times, working memory, and a more clear-headed, composed, and energetic experience. Riboflavin (vitamin B2) is necessary for the mitochondrial processes that generate cell energy as ATP, which the brain uses intensely. Riboflavin is also needed to activate and utilize several other brain-essential vitamins (e.g., vitamins B3, B6, and B9) and plays a role in supporting the function of one of the body's major antioxidants and detoxification molecules—glutathione. Niacinamide, a form of vitamin B3, is a precursor to a molecule called NAD, which plays a central role in ATP production, cellular defense functions, cellular repair and stress response functions, and several pathways linked to healthier aging. NAD and other vitamin B3-dependent molecules are essential for healthy brain function and nerve health, with mental confusion being one of the signs of vitamin B3 deficiency. Pantothenic acid (vitamin B5) is a cofactor in biosynthetic pathways that support cellular energy metabolism and the synthesis of the neurotransmitter acetylcholine. Therefore, pantothenic acid supports mental energy and brain functions associated with acetylcholine, such as alertness, sustained attention, learning, and memory. Pyridoxal 5′-phosphate (P5P) is a coenzyme or active form of vitamin B6. P5P is involved in over 4% of all enzymatic activities, including many metabolic pathways important for cellular energy generation and the production of neurotransmitter molecules involved in motivation and mood, including dopamine, catecholamines, and serotonin. Folate (vitamin B9) is considered a brain-essential vitamin since it's needed for myelin production, nerve function, and the production of several neurotransmitters (e.g., serotonin, melatonin, dopamine, and norepinephrine). It complements the action of other B vitamins, especially B6 and B12, in supporting neurological function and brain health. Methylcobalamin (vitamin B12) plays a role in converting fats and sugars into energy, thereby supporting cellular energy production in the brain. It is also essential for supporting the healthy function of brain and nerve cells, brain volume, mood, and memory.

The present composition may be administered orally. In such an embodiment, the mental energy support aid may be integrated into a liquid, capsule, tablet, chewable tablet, or pill. In an exemplary embodiment, the composition is integrated into capsules and taken orally with breakfast and/or lunch.

The composition works unexpectedly well in that by using multiple ingredients that support brain energy production and a wide range of brain and nervous system functions that are needed for supporting the different dimensions of mental energy, the composition has a more robust effect in boosting the sense of mental energy during the day, manifesting as increased alertness, sustained attention, focus, mood, and motivation. The composition further works unexpectedly well in that by taking advantage of caffeine's actions in supporting alertness, energy, and motivation but combining it with other compounds that counter the jitteriness and irritability caffeine may cause and that prevent a caffeine crash after a few hours, the composition amplifies and extends the mental energy and productivity benefits typical of caffeine. The constituents form a synergistic compound where the combined effect is greater than the sum of their separate effects.

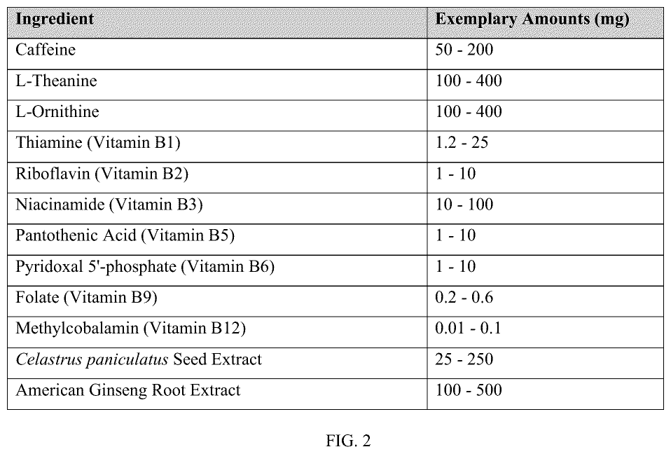

In an exemplary embodiment of the present invention and with reference to FIG. 2, the composition further comprises one or more of the following ingredients: Celastrus paniculatus Seed Extract and American Ginseng (Panax quinquefolius) Root Extract. In one embodiment of the invention, the total mg in one dose is 691 mg and is taken in two capsules (345.5 mg/capsule). Celastrus paniculatus is native to India, where it's used by local healers primarily as a brain tonic for reasons consistent with one of its common names, “the intellect tree.” These uses support brain performance in mental acuity, memory, mental fatigue, and stress. It was believed by native healers that people eating the seeds of this plant would be able to learn new information more quickly and accurately and efficiently recall later. Celastrus may support mental sharpness and counter mental fatigue by contributing to the support of attention, focus, energy, and motivation. American Ginseng (Panax quinquefolius) is the same genus as Panax ginseng and is prized for the same reasons: both are considered adaptogens that help support resistance to various stressors. As an adaptogen, American Ginseng root extract is used as an energy tonic in times of higher stress or greater demands to support the capacity for mental and physical work. As a nootropic, American Ginseng extracts support working memory (a cognitive skill in the executive function category) and a calm mood.

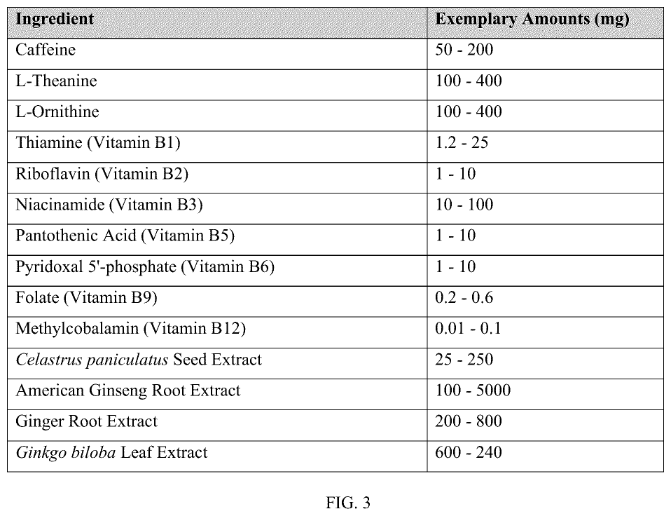

In an exemplary embodiment of the present invention and with reference to FIG. 3, the composition further comprises one or more of the following ingredients: Ginger Root Extract and Ginkgo biloba Leaf Extract. In one embodiment of the invention, the total mg in one dose is 1211 mg and is taken in two capsules (605.5 mg/capsule). Ginger has a long history of use as a food spice and in traditional healing systems, including Traditional Chinese Medicine (TCM) and Ayurveda. Although it's typically not considered a nootropic, ginger supports the brain through continued use over extended periods. It supports attention, working memory, and cognitive processing efficiency. Ginger promotes healthy blood sugar regulation, essential for brain energy production. Ginger also supports the gut-brain axis and neuroprotection functions and complements Ginkgo biloba in supporting mood. Ginkgo biloba is one of the most widely used herbs for brain health. Ginkgo extracts may help a number of functions involved in healthy brain function, including supporting several neurotransmitter systems and neurogenesis while promoting healthy vascular function and blood flow to the brain. Ginkgo is best known for supporting cognitive skills related to attention, concentration, memory, and mood. Its functions complement those of Ginger and American Ginseng.

In addition to the exemplary ingredients disclosed, other components may be used to assist administration. The composition may also include binding agents or other inactive ingredients in an example where the mental energy aid is integrated into a capsule or tablet.

The present composition may be administered orally. In such an embodiment, the mental aid may be integrated into a liquid, capsule, tablet, chewable tablet, or pill. The composition is integrated into capsules and taken orally with breakfast or lunch in an exemplary embodiment. In such an embodiment, a dietary supplement to improve mental energy may be prepared by first combining Caffeine, L-Theanine, L-Ornithine, and B-complex vitamins—Thiamine (B1), Riboflavin (B2), Niacinamide (B3), Pantothenic Acid (B5), Pyridoxal 5′-phosphate (B6), Folate (B9), Methylcobalamin (B12) (as exemplary ingredients) and mixing until a homogeneous mixture is produced. The composition may then be integrated into capsules to be taken orally.

The present disclosure may also be administered to a person in other ways without departing from the embodiments contemplated herein, including pressing into a tablet form, including in a food or beverage, and other methods of oral ingestion.

To make the composition, one takes the ingredients described in the exemplary embodiments and mixes them in powder form until a homogeneous mixture is achieved. The composition is then packaged into capsules to be taken orally.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein but instead as being fully commensurate in scope with the following claims.

1. A composition comprising Caffeine, L-Theanine, L-Ornithine, and B-complex vitamins-Thiamine (B 1), Riboflavin (B2), Niacinamide (B3), Pantothenic Acid (B5), Pyridoxal 5′-phosphate (B6), Folate (B9), and Methylcobalamin (B12).

2. The composition of claim 1 further comprising Celastrus paniculatus Seed Extract, American Ginseng (Panax quinquefolius) Root Extract, or a combination thereof.

3. The composition of claim 2 further comprising Ginger Root Extract, Ginkgo biloba Leaf Extract, or a combination thereof.

4. The composition of claim 1, wherein the composition is integrated into a capsule, tablet, chewable tablet, or pill.

5. The composition of claim 1, wherein the composition is integrated into a liquid or powder.

6. A method for boosting the sense of mental energy during the day, manifesting as increased alertness, attention, focus, mood, and motivation, by promoting brain energy production and a wide range of brain and nervous system functions that support mental energy, the method comprising the steps of:
ingesting a composition comprising Caffeine, L-Theanine, L-Ornithine, and Bcomplex vitamins-Thiamine (B 1), Riboflavin (B2), Niacinamide (B3), Pantothenic Acid (B5), Pyridoxal 5′-phosphate (B6), Folate (B9), and Methylcobalamin (B12).

7. The method of claim 6, wherein the composition further comprises Celastrus paniculatus Seed Extract, American Ginseng (Panax quinquefolius) Root Extract, or a combination thereof.

8. The method of claim 7, wherein the composition further comprises Ginger Root Extract, Ginkgo biloba Leaf Extract, or a combination thereof.

9. The method of claim 6, wherein the composition is integrated into a capsule, tablet, chewable tablet, or pill.

10. The method of claim 6, wherein the composition is integrated into a liquid or powder.
